(19) United States
(12) Patent Application Publication
(10) Pub. No.: US 2011/0002844 A1
(43) Pub. Date: Jan. 6, 2011

(54) STABILIZATION OF MACROMOLECULAR MEMBRANES

(75) Inventors: Joshua S. Katz, Philadelphia, PA (US); Jason A. Burdick, Philadelphia, PA (US); Daniel A. Hammer, Villanova, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Trustees of the University of Pennsylvania, Philadelphia, PA (US)

(21) Appl. No.: 12/919,119

(22) PCT Filed: Feb. 11, 2009
(86) PCT No.: PCT/US09/33722

§ 371 Date: Sep. 16, 2010

Related U.S. Application Data
(60) Provisional application No. 61/037,087, filed on Mar. 17, 2008.

(51) Int. Cl. A61K 51/06; A61K 9/50; A61K 31/7088; A61K 38/02; A61K 31/7105; A61K 31/711; A61K 49/00; A61K 31/704; A61P 35/00

(52) U.S. Cl. 424/1.21; 424/497; 514/44 R; 514/1.1; 424/9.1; 514/34

(57) ABSTRACT

Disclosed are stabilized polymersomes having layer structures. Such stabilized polymersomes are, in some embodiments, biocompatible, and are capable of enhanced, sustained release of agents. Also disclosed are related methods for synthesizing such stabilized polymersomes and methods for using such polymersomes for delivery of therapeutic, imaging, and various other agents.

### RELATED APPLICATIONS

This application claims priority to U.S. Application Ser. No. 61/037,087, filed on Mar. 17, 2008, the entirety of which is incorporated herein by reference.

### STATEMENT OF GOVERNMENT INTEREST

The government may have certain rights in the claimed invention. This work was supported by National Science Foundation grant NSF DMR-05-20020.

### FIELD OF THE INVENTION

The instant invention relates to the fields of polymer chemistry and polymeric vesicles. The instant invention also relates to the field of controlled drug delivery.

### BACKGROUND OF THE INVENTION

There has recently been a surge in research in the fabrication of improved drug delivery devices for the treatment of a variety of diseases, including as cancer therapies. Polymersomes are one class of drug delivery vehicles that offer promise for in vivo applications because of their robust stability and high payload capacity.

There is, however, a need in the art for polymersomes capable of targeted drug delivery of a particular agent or agents to specific locations in the body while also minimizing systemic release of such agents. The value of such polymersomes would be further enhanced if the polymersomes were capable of controlled, sustained release of therapeutic and other agents.

### SUMMARY OF THE INVENTION

In meeting the described needs in the art, the present invention first provides methods of synthesizing stabilized polymersomes, comprising forming a polymersome, having a layer structure, from chains of multiblock copolymer comprising hydrophobic and hydrophilic blocks; at least some of the hydrophobic blocks comprising one or more polymerizable groups; and reacting two or more of the polymerizable groups to form covalent bonds between chains of the copolymer.

In a second aspect, the claimed invention provides stabilized polymersomes, comprising a polymersome comprising a multiblock copolymer layer structure, the layer structure comprising multiblock copolymer chains having hydrophilic and hydrophobic blocks, and two or more chains being covalently bonded to one another.

The claimed invention further provides methods for delivering an agent to a subject, comprising introducing one or more stabilized polymersomes into a patient, the one or more stabilized polymersomes comprising a layer structure comprising multiblock copolymer, the multiblock copolymer comprising hydrophobic and hydrophilic blocks, the one or more stabilized polymersomes comprising one or more therapeutic agents; and exposing the one or more stabilized copolymers to a stimulus to effect release of the one or more therapeutic agents.

Also provided are methods for altering the properties of a polymersome, comprising providing a polymersome, the polymersome comprising a multiblock copolymer layer structure, the structure comprising a plurality of multiblock copolymer chains, the multiblock copolymer comprising hydrophilic and hydrophobic blocks; and forming covalent bonds between two or more hydrophobic blocks of multiblock copolymer chains.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1 illustrates polymersomes loaded with sucrose and imaged in isoösmolar PBS. A. No DMPA. no UV. B. No DMPA. 30 min UV. C. DMPA. no UV. D. DMPA, 30 min UV. Scale bar=20 microns;

FIG. 2 illustrates monitoring of DOX release from polymersomes, (A) shows fluorescence scans of one sample of acrylate-stabilized polymersomes containing DOX over time (0, 8, 24, 48, 120 hrs; direction of arrow) and (B) shows the ratio of acid to neutral fluorescence after background subtraction for unmodified polymersomes (squares) and acrylate-stabilized polymersomes after UV exposure (triangles);

FIG. 3 illustrates a non-limiting selection of candidate hydrophobe chemistries of block-co-polymer formulations;

FIG. 4 illustrates an exemplary, non-limiting scheme for placing reactive, polymerizable groups (e.g., acryl) onto polymer chains;

FIG. 5 depicts an exemplary, non-limiting scheme for placing DOX into the interior of an exemplary polymersome;

Scheme 1 depicts an exemplary synthesis route for production of acrylate-terminated PCL-PEG copolymer;

Scheme 2 is a schematic of hydrophobic end group polymerization for stabilizing polymersome membranes, which scheme includes UV-light based induction of radical polymerization through functional groups of the inventive polymer compositions;

FIG. 6 illustrates NMR spectra of the disclosed compositions—lowercase letters indicate assignment of peaks to the chemical structure shown, (A) NMR spectra of dehydrated polymersomes of AcPCL-PEG with or without DMPA loaded into the membrane before and after UV light exposure as indicated; the −DMPA+UV sample received a 30 minute dose of UV light, while the +DMPA+UV sample received a 5 minute dose, (B) NMR spectra of AcPCL-PEG polymersomes with varying amounts of DMPA loaded into the membrane—all samples received a 10 minute dose of UV light;

FIG. 7 depicts (a) GPC traces of reconstituted polymersomes containing varying amounts of DMPA in the membrane and exposed to UV light (the GPC traces include only the soluble portions of the THF samples), and (b) The percentage of polymer that remained insoluble during reconstitution in THF;

FIG. 8 depicts (a) CryoTEM images of AcPCL-PEG polymersomes without initiator or light (left), with initiator alone (center), or with both initiator and UV light exposure (right), indicating that the polymersome morphology is not affected by initiator or light (scale bars=100 nm), and (b) DLS intensity distributions of polymersomes loaded with DMPA before (solid line) and after (dashed line) UV exposure;

FIG. 9 depicts fibroblast viability when cultured in the presence of polymersomes consisting of PCL-PEG (black), AcPCL-PEG (dark grey), AcPCL-PEG+DMPA (light grey), or AcPCL-PEG+DMPA+UV (white) for (a) 24 and (b) 72 hours. All samples (n=3) are normalized to cultures without polymersomes present (* p<0.05);

FIG. 10 depicts self-assembled polymer morphologies before (solid lines) and after (dashed lines) treatment with Triton X-100 (available from, e.g., Sigma-Aldrich, www.sigma.com) for DMPA-loaded polymersomes (a) before and (b) after UV exposure;

FIG. 11 depicts DLS of polymersomes before (-UV) and after (+UV) exposure initially (solid line) or when incubated in acetate buffer for 12 days (dashed line);

FIG. 12 depicts (a) cumulative % released and (b) release rates of DOX encapsulated in AcPCL-PEG polymersomes with 1:1 DMPA either without exposure (circles) or exposure to 15 minutes UV light (squares)—the amount released was normalized to the initial amount encapsulated and is reported as means (n=3) and standard deviations; and

FIG. 13 depicts cellular response to treatment with DOX-loaded polymersomes—total DOX dose was 12.2 mg/mL (high) or 1.22 mg/mL (low) with or without UV-stabilization, and viability was measured in comparison to PBS controls at 24 (black), 48 (grey), and 72 (white) hours; data are reported as n=3 and standard deviations (* p<0.05 for the indicated groups, and all groups saw significant (p<0.05) changes between the 24, 48, and 72 hour time points).

### DETAILED DESCRIPTION AND ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

In one embodiment, the claimed invention provides methods of synthesizing stabilized polymersomes. These methods include, inter alia, forming a polymersome, having a layer structure, from chains of multiblock copolymer comprising hydrophobic and hydrophilic blocks; at least some of the hydrophobic blocks comprising one or more polymerizable groups; and reacting two or more of the polymerizable groups to form covalent bonds between chains of the copolymer.

The multiblock copolymer may include a diblock copolymer, a triblock copolymer, and combinations thereof. Tetra- and higher block copolymers may be used. Poly(caprolactone)-poly(ethylene glycol) is considered especially suitable, although other block copolymers that include hydrophobic and hydrophilic blocks may be used. Exemplary diblock copolymers having hydrophobic and hydrophilic blocks include poly(ε-caprolactone)-poly(ethylene oxide), and poly(ε-caprolactone)-poly(ethylene glycol); poly(ε-caprolactone)-poly(ethylene glycol) is considered especially suitable because poly(ethylene glycol) (known as “PEG”) is known to be essentially invisible to the immune system, and is thus useful in applications where a polymersome is delivered to a living subject. Other suitable copolymers are described elsewhere herein.

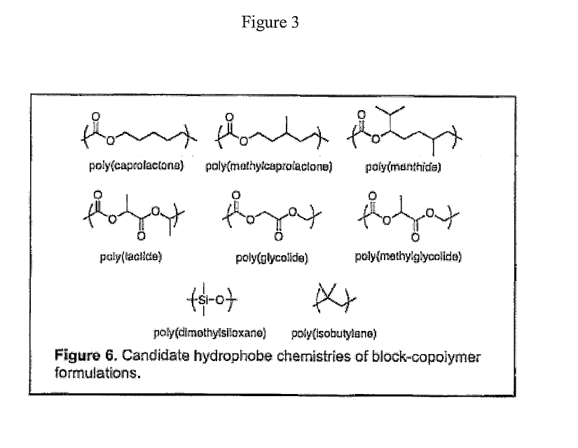

Such copolymers may be of an X-Y (hydrophobic-hydrophilic) configuration, where layers of the polymer form bilayers. In other embodiments, the copolymers may be of an A-B-A configuration, where the A is a hydrophilic block, and B is a hydrophobic block. At least some of the hydrophobic blocks suitably bear one or more polymerizable groups; a non-exhaustive list of hydrophobic block candidates is shown in FIG. 3, and variations on these candidates are also within the scope of the claimed invention.

The reacting suitably comprises reacting two or more of the polymerizable groups to form covalent bonds between different blocks of the copolymer. This may be accomplished in the presence of an initiator. Suitable polymerizable groups include acryl groups and other groups described elsewhere herein. As will be apparent to those of skill in the art, a variety of groups capable of polymerization will be useful in the claimed compositions. The compositions may include polymerizable groups of one kind of multiple kinds

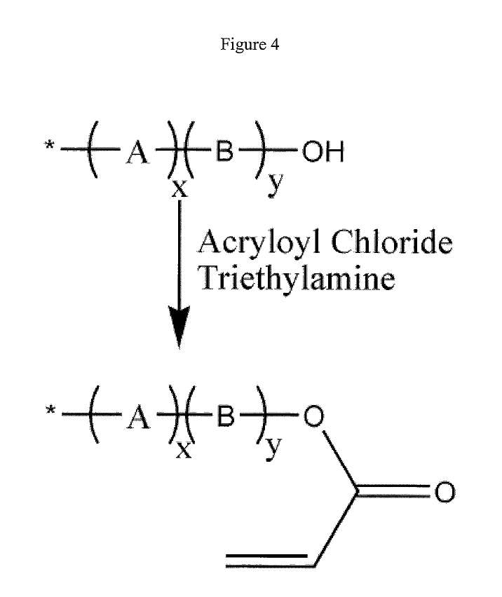

Polymerizable groups may be present on the polymer ab initio, but in some embodiments may be grafted onto or otherwise included in the hydrophobic blocks during the course of the synthesis. Suitable methods for performing such inclusion will be known to those of ordinary skill in the art; one such method is shown in FIG. 4. That figure illustrates acrylation of the —OH end of a block copolymer, where A is a hydrophilic block, and B is a hydrophobic block.

The reacting is, in some embodiments, effected by an environmental condition exterior to the multiblock copolymer, which condition can be an acidic condition, a basic condition, heat, cold, and the like. Reacting the polymerizable groups is accomplished by, for example, exposing the polymerizable groups to ultraviolet light. This is suitably performed in the presence of a UV initiator, such as 2,2-dimethoxy-2-phenylacetophenone (DMPA).

Other means of reacting the polymerizable groups so as to form covalent bonds will be apparent to those of ordinary skill in the art, and, in some embodiments, may include reaction that is chemically driven or reaction that is effected by exposure to radiation. In some embodiments a comparatively small fraction of the hydrophobic blocks bear one or more polymerizable groups; in others, at least a majority of the hydrophobic blocks bear one or more polymerizable groups. The degree to which the hydrophobic blocks bear polymerizable groups will be dictated by the needs of the user, and the present invention is not limited to embodiments having a particular amount of polymerizable groups being present. Reaction may be performed in the presence of an initiator and an environmental stimulus.

The layer structure of the disclosed polymersomes is, in some embodiments, a bilayer. In other embodiments, the polymersome comprises a monolayer structure. In the bilayer embodiment, covalent bonds may be formed between the inner and outer layers of the bilayer.

Acrylates, methacrylates, acrylamides, methacrylamides, vinyls, and vinyl sulfone units are all suitable polymerizable groups. Groups that yield biocompatible products when reacted are considered suitable, although biocompatibility is not required. The polymersomes may include a wide range of polymerizable groups, and the materials used to make up a polymersome may include two or more kinds of polymerizable groups. The polymerizable groups may be present in a wide range, and may be present from about 0 wt % up to about 100 wt %.

The optimal proportion of polymerizable groups to polymer and to initiator will be determined by the user; in some cases, a majority of hydrophobic blocks bear polymerizable groups; in others, less than half of the hydrophobic blocks bear polymerizable groups. In suitable embodiments, the copolymer is chosen to as to result in the stabilized polymersomes being essentially biodegradable. In other embodiments, the copolymer is chosen to as to result in the stabilized polymersomes being partially biodegradable, or even non-biodegradable.

One or more agents may be disposed within the stabilized polymersome, within the layer structure, on the surface of the polymersome, or any combination thereof. As one non-limiting example, an imaging agent may be disposed within the interior of the polymersome. In another embodiment, a ligand or other agent may be disposed on the surface of the polymersome so as to enable the polymersome to bind—uniquely—to a particular receptor or other target. An agent may be a therapeutic composition, an imaging composition, a binding composition, a drug, a therapeutic compound, a nanoparticle, an imaging agent, a contrast agent, a nutrient, a vitamin, a protein, DNA, RNA, an oligonucleotide, a salt, a gene, a biological material, a magnetic material, a radioactive material, and the like. Agents present on the surface of a polymersome are suitable coupled or otherwise bound to the polymersome.

Agents—such as antibodies, antigens, ligands, and receptors—that effect binding of a polymersome to a cell or other biological tissue are also suitable. Combinations of agents may be used. As one example, polymersomes according to the claimed invention may include an imaging agent and a therapeutic agent so as to enable a polymersome have double functionality.

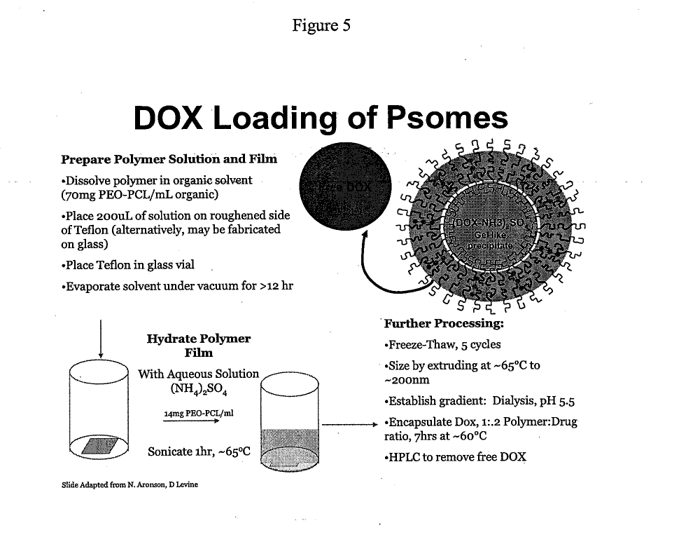

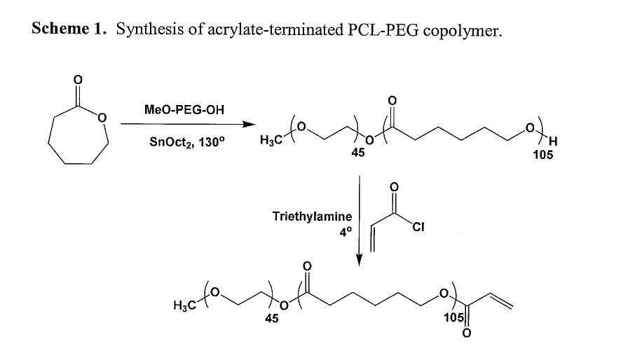

One suitable method for disposing the one or more agents in a polymersome is dialysis, an example of which is shown in FIG. 5. Other methods for disposing or encapsulating agents within the polymersomes are known to those of ordinary skill in the art.

One or more crosslinks between multiblock copolymer chains may be formed. These cross-links are suitably formed by introducing a composition having multiple polymerizable groups to the chains of multiblock copolymer, although in some cases, the multiblock copolymer itself includes multiple polymerizable groups. Polymersomes made according to the disclosed methods are within the scope of the claimed invention.

The cross-links may be formed by use of a diacrylate or other reactive species, which species may be doped into the membrane of the disclosed compositions, or, in other embodiments, is present on the polymer chains used in the polymersome. Cross-linking between chains of a membrane suitably enhances the rigidity of the polymersome composition, but the composition may degrade more slowly than a composition that is merely stabilized—as opposed to stabilized and cross-linked. Depending on the needs of the user, a cross-linked, stabilized membrane may be suitable for certain applications where particular mechanical properties are desired. Cross-linked polymersomes may be non-biodegradable, which may be useful in applications where the user desires that the polymersomes remain for an extended period of time.

The present invention also provides stabilized polymersomes. These polymersomes include a polymersome comprising a multiblock copolymer layer structure, the layer structure comprising multiblock copolymer chains having hydrophilic and hydrophobic blocks, and two or more chains being covalently bonded to one another. According to the claimed invention, two or more hydrophobic blocks are suitably covalently bonded to one another, as shown in Scheme 2, which figure shows a bilayer structure wherein hydrophobic blocks are covalently bound to one another.

The layer structure is preferably a bilayer, although the inventive polymersomes may also have one or multiple layers. Suitable multiblock copolymers for the polymersomes are described elsewhere herein, and the polymersomes suitably include two—or more—hydrophibic blocks covalently bonded to one another.

The polymersomes are suitably biodegradable. Biodegradability is accomplished by, for example, polymersomes comprising polycaptolactone and poly-ethylene glycol. Other hydrophilic and hydrophobic blocks that are also known to be biodegradable may be incorporated into the disclosed polymersomes.

Suitably copolymers used in the polymersomes include polymers wherein a hydrophilic block comprises poly(ethylene oxide), poly(acrylic acid), poly(ethylene glycol), and the like. The hydrophobic block may include, for example, poly(caprolactone), poly(methylcaprolactone), poly(menthide), poly(lactide), poly(glycolide), poly(methylglycolide), poly(dimethylsiloxane), poly(isobutylene), poly(styrene), poly(ethylene), poly(propylene oxide), and the like.

One or more agents may be disposed within the layer structure, within the interior of the polymersome, on the surface of the polymersome, and the like. An agent may be, for example, a drug, a therapeutic compound, a nanoparticle, an imaging agent, a contrast agent, a nutrient, a vitamin, a protein, DNA, RNA, an oligonucleotide, a salt, a gene, a biological material, a magnetic material, a radioactive material, and the like. Chemotherapy agents are considered suitable, as are analgesics.

The agent is suitably disposed in some embodiments within the interior of the polymersome. In other embodiments, the agent is disposed within the membrane—e.g., bilayer—of the polymersome. In still other embodiments, agents are disposed within the interior of the polymersome and within the membrane. As will be apparent to those of ordinary skill in the art, agents may suitable be chosen depending on the desired locus of disposition.

The polymersomes are also capable of releasing the aforementioned agents. In some embodiments, an agent disposed within the polymersome, within the layer structure of the polymersome, or both, is released at a higher rate when the stabilized polymersome is exposed to a stimulus. Stimuli include heat, acid, basic conditions, light, radiation, osmotic gradients, oxidative or reductive stresses, and the like. As one non-limiting example, the user concentrates the stimulus to a particular location in a subject so as to enhance release of agents from any polymersomes disposed at that location. In another example, the polymersomes are delivered to a subject wherein target areas within the subject exhibit one or more stimuli that effect enhanced release of agents from polymersomes. In these embodiments, the polymersomes are capable of effecting enhanced agent delivery at those locations in a subject where agent delivery is most desired.

The polymersomes may include one or more cross-links between one or more multiblock copolymer chains. Those of ordinary skill will encounter no difficulty in forming cross-links between copolymer chains, which cross-links may be formed, for example, between a di-acrylate and reactive groups on copolymer chains.

In embodiments wherein the polymer chains of the polymersomes are crosslinked, a portion of the block copolymer is then removed—by dissolution, for example—so as to leave behind a shell comprised of the remaining, cross-linked portion of the polymersome. As one non-limiting example, a bilayer polymersome is formed from poly(caprolactone)-poly(ethylene glycol) block copolymer. Crosslinks are formed between the poly(caprolactone) terminus and a diacrylate. The poly(caprolactone)-poly(ethylene glycol) is then removed so as to leave behind a shell of the crosslinked diacrylate. In this way, ultra-thin shells comprising hydrophobic compositions may be made and used for agent delivery or imaging applications.

The cross-linking may be accomplished by, for example, introducing one or more diacrylates. Suitable diacrylates include, for example 1,4-butane diol diacrylate, ethylene glycol diacrylate, and oligo-β-aminoesters, The multi-acrylates (di-, tri-, tetra- and higher) acrylates can be loaded into the polymersome as is done, for example, with DMPA or other initiators. In other embodiments, the multi-acrylate is covalently attached to the polymer. Either—or both—of these methods for incorporating multi-acrylates into the are suitable for cross-linking polymersomes according to the claimed invention.

The invention also provides methods for delivering an agent to a subject. These methods include introducing one or more stabilized polymersomes into a patient, the one or more stabilized polymersomes comprising a layer structure comprising multiblock copolymer, the multiblock copolymer comprising hydrophobic and hydrophilic blocks, the one or more stabilized polymersomes comprising one or more therapeutic agents; and exposing the one or more stabilized copolymers to a stimulus to effect release of the one or more therapeutic agents.

The methods are suitably applied such that the one or more stabilized polymersomes are taken up by one or more cells characterized by a diseased state. The polymersomes may be delivered by injection, ingestion, or by other methods known in the art to deliver agents to subjects. The ability of the claimed polymersomes to release agents upon exposure to specific environmental stimuli enables creation of delivery systems that are specific to a particular environmental conditions that are present at specific locations or targets.

In some embodiments, diseased cells expose the one or more stabilized polymersomes to the stimulus. For example, the polymersomes may be designed such that the environment exterior to the cells—or inside the cells—enhances degradation of the polymersomes so as to effect enhanced release of one or more agents in the vicinity—or inside of—the cell. The delivered polymersomes may be used to treat diseases, to image parts of a subject, or both.

The claimed invention also provides methods for altering the properties of a polymersome, which methods include providing a polymersome, the polymersome comprising a multiblock copolymer layer structure, the structure comprising a plurality of multiblock copolymer chains, the multiblock copolymer comprising hydrophilic and hydrophobic blocks; and forming covalent bonds between two or more hydrophobic blocks of multiblock copolymer chains. The formation of the covalents is accomplished by polymerizing reactive groups present on the hydrophobic blocks, which process is described elsewhere herein. In some embodiments, one or more cross-links are formed between copolymer chains. In some embodiments, the layer structure of the polymersome being modified includes a bilayer, although the polymersome can be a monolayer.

These methods are useful, e.g., in modifying the characteristics of a polymersome so as to alter its release profile. For example, one may stabilize a polymersome so as to slow the rate at which th polymersome degrades and releases an agent disposed within. In other embodiments, the polymersome may be stabilized so as to strengthen the polymersome such that the polymersome is more mechanically durable. Such modifications may be useful where a non-stabilized polymersome degrades too quickly—e.g., if it were exposed to turbulent or fast fluid flow—for the user's needs. By stabilizing the polymersome, the polymersome is newly capable of withstanding mechanical stresses that would disrupt or adversely affect a non-stabilized polymersome.

As a non-limiting example, polymersomes may be administered to a cancer patient. The polymersomes travel through the neutral environment of the patient, releasing only a minimal amount of any agents contained within the polymersomes. The polymersomes are then taken up by a cancerous cell, and upon exposure to the acidic environment within or near to the cancerous cell, the polymersomes release their contents into—or in the vicinity of—the cancerous cell. In such an embodiment, the polymersome minimizes the release of its agent into the patient's system while traveling to the target cancer cell, and instead conserves that agent until the polymersome is taken up by the acidic cancer cell, where it releases the therapeutic agent. In this—and other embodiments—the advantages of a stabilized polymersome over a non-stabilized polymersome become clear because the stabilized polymersome releases its contents in a controlled, delayed fashion over a period of time, rather than in an initial burst that releases a large proportion of the polymersome's contents. A stabilized polymersome can, depending on the user's needs, likewise have an advantage over a cross-linked polymersome because the stabilized polymersome will degrade—and release its contents—more slowly than a cross-linked polymersome, and certain cross-linked polymersomes may not be biocompatible or biodegradable.

The stabilized polymersomes of the present invention are suitably characterized as being essentially biodegradable. Both polymersomes that degrade of their own accord over time and polymersomes that degrade in response to a stimulus—such as heat, acidic conditions, basic conditions, and radiation—are within the scope of the claimed invention. Likewise, biocompatible, non-biodegradable polymersomes may also be synthesized according to the claimed invention. Such polymersomes may be useful where the relatively long-term presence of a polymersome is useful, for example, where it is necessary to image a portion of a subject over an extended period of time.

Hydrophilic blocks suitable for the claimed invention include poly(ethylene oxide), poly(acrylic acid), poly(ethylene glycol), and the like. Other hydrophobic blocks suitable for inclusion in the claimed polymersomes will be apparent to those of ordinary skill in the art.

Suitable hydrophobic blocks include comprises poly(caprolactone), poly(methylcaprolactone), poly(menthide), poly(lactide), poly(glycolide), poly(methylglycolide), poly(dimethylsiloxane), poly(isobutylene), poly(styrene), poly(ethylene), poly(propylene oxide) and the like. Suitable reactive endgroups include acrylates, methacrylates, methacrylamides, vinyls, vinyl sulfones, and acrylamides.

### EXPERIMENTAL RESULTS AND NON-LIMITING EMBODIMENTS

The following section describes non-limiting results achieved by illustrative embodiments of the claimed invention. These results are illustrative only and do not necessarily limit the scope of the claimed invention.

### First Example Embodiment

In one illustrative embodiment, it was shown that at physiological pH, DOX is initially released by passive diffusion across the polymersome membrane, followed by a faster release as the membrane hydrolyzed. At endosomal pH, however, release was completely mediated by membrane hydrolysis. To minimize the systemic release of a drug from polymersomes for cancer therapies, it was sought to decrease the passive diffusion of DOX by altering the membrane structure.

To this end, the terminal hydroxyl endgroup of the PCL block of the polymer was acrylated prior to polymersome formation. While acryl groups were used in this work, other suitable polymerizable groups will be known to those of ordinary skill in the art.

With the addition of an initiator and light source, the acrylate underwent free-radical polymerization (confirmed with ¹H NMR) and stabilized polymersome membrane. Indeed, this procedure decreased the early release of DOX from polymersomes at physiological pH and did not affect the overall polymersome morphology.

### Experimental

Materials. Except where noted, all materials were used as received. Acryloyl chloride, triethylamine (TEA, anhydrous grade), poly(ethyl ene glycol) methyl ether (PEO, Mn=2,000 Da), stannous octoate, ε-caprolactone (CL), 2,2-dimethoxy-2-phenylacetophenone (DMPA) and DOX were received from Sigma Aldrich. CL was dried for 48 hours over calcium hydride and distilled prior to use. PEO was dried under vacuum at 90° C. overnight before use. All solvents were reagent grade or better.

Instrumentation. All ¹H-NMR spectra were recorded on a Bruker Avance 360 MHz NMR machine in deuterated chloroform. GPC was run on a Waters GPC calibrated to polystyrene standards at 5 mg/mL in THF. For UV exposure, a spot-curing UV lamp with a collimating lens was employed (365 nm, ˜55 mW/cm², 5 cm from sample. Omnicure Series 1000, Exfo, Quebec, Canada). Microscope images were taken using a Zeiss Axiovert 200 microscope. Fluorescence spectra were obtained using a Tecan Infinite 200 plate-reader in 96-well blackwell plates.

Synthesis of Polymers. PCL-PEO was synthesized by a standard ringopening polymerization of CL using PEO as a macroinitiator and stannous octoate as a catalyst. Briefly, 2.0 g PEO was added to 12g CL (11.6 5 mL) in a 100 mL round-bottomed flask under an argon atmosphere. 12 drops of stannous octoate were added, and the flask was sealed. The reaction was first heated to 90° C. for 30 minutes to fully dissolve the PEO in the CL, followed by heating to 130° C. for 1.5 hrs while under vacuum. The crude polymer was dissolved in THF, precipitated into hexanes, and dried.

The hydroxyl terminus of the PCL was acrylated by reacting with acryloyl chloride and TEA. 5.2 grams of PCL-PEO was dissolved in 200 mL dichloromethane (DCM) under a stream of argon in a 500 mL 3-neck roundbottomed flask equipped with an addition funnel. 500 uL (˜10×) TEA was injected into the flask and the temperature was reduced to 0° C. The addition funnel was charged with 300 uL (˜10×) acryloyl chloride and 30 mL DCM. The acryloyl chloride solution was added dropwise to the reaction vessel over 1 hour. The reaction was allowed to proceed for 4 hrs at 0° C. followed by overnight at room temperature. Pure polymer was recovered by concentration, dissolution in benzene (to precipitate triethylammonium salts), filtration, reconcentration in DCM, precipitation into hexanes, and drying.

Polymers were characterized for their number average molecular weight by ¹H-NMR spectroscopy and for polydispersity by GPC.

Fabrication of Polymersomes. Polymersomes were fabricated by hydration of thin-films of polymer on roughened Teflon. Films were solvent cast out of DCM at either 16 mg/mL (for giant polymersomes) or 70 mg/mL (for nanoscale polymersomes). Samples containing DMPA were made by cocasting an equimolar amount of DMPA with the acrylated PCL-PEO (AcPCL-PEO, 18 ug/mg polymer). Films were hydrated with 2 mL of aqueous media (water for NMR study, 290 mOsM sucrose for giant polymersomes, and 290 mOsM ammonium sulfate (pH=5.4) for DOX loading). Giant polymersomes were assembled by incubating the hydrated films for 48 hrs at 65° C. followed by vortexing for one min. Nanoscale polymersomes were equilibrated at 65° C. for 30 minutes following hydration and sonicated for 60 min.

DOX release. Following sonication, polymersomes for DOX release studies were cycled through freezing in liquid nitrogen and thawing at 65° C. five times followed by extrusion through 400 nm (1×) and 200 nm (3×) membranes. Following dialysis against isoosmolar sodium acetate (pH=5.5, 3 exchanges over 18 hours), the polymersomes were incubated with DOX (0.2 g DOX/g polymer) for 9 hours at 65° C. Free DOX was removed by separation on a HITRAP desalting column (GE Healthcare), and polymersomes were diluted into PBS (pH 7.4 or 5.5) (see FIG. 5). UV-exposed samples were irradiated for 5 minutes, and then samples were aliquoted into 96 well plates. Release was monitored fluorometrically with excitation of 480 nm.

Results and Discussion

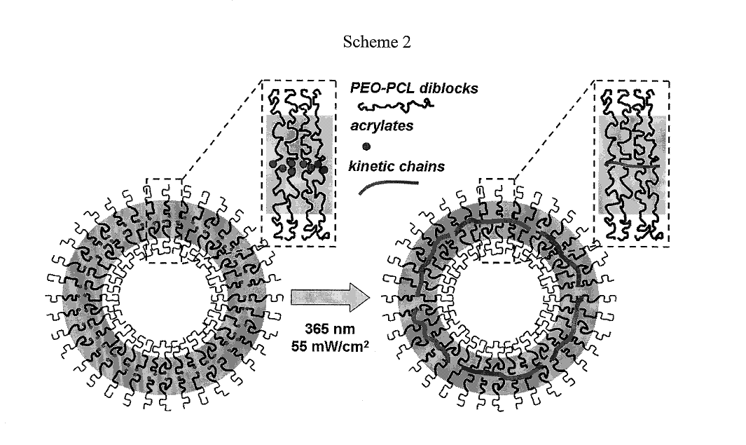

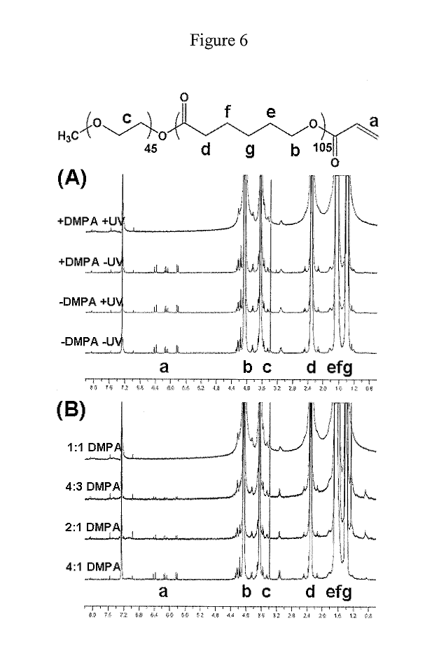

Synthesis of PCL-PEO and AcPCLPEO. PCL-PEO was synthesized according to standard procedures using stannous octoate as the catalyst. The resulting polymer was found to have a number average molecular weight of ˜14 kDa (˜12 and ˜2 kDa for the PCL and PEO blocks, respectively). This was determined by calibrating the NMR peaks to the terminal methoxy group on the PEO at approximately 3.4 ppm (FIG. 6). The polydispersity of the polymer was 1.25. Acrylation of the OH terminus of the PCL block did not lead to a significant change in the polymer size or distribution following the second purification. The acrylation efficiency was found to be 99%.

Acrylate Polymerization within Polymersome Membranes. When polymersomes self-assemble, the polymer chains align in a bilayer structure such that the inner and outer surfaces of the membrane are coated with the PEO, while the inner part of the membrane contains the hydrolytically-degradable PCL. Because of the high crystallinity of the PCL portion of the polymer, at physiological temperature, the membrane is rigid, and the acrylates should be localized to the center of the bilayer. When DMPA was loaded into the membrane, it was evenly dispersed within the hydrophobic portion, so not all of it was localized to initiate acrylate polymerization.

To confirm that acrylate conversion was occurring within the polymersome membrane as a result of DMPA photoinitiation, nano-scale polymersomes were synthesized from AcPCL-PEO in water, with or without DMPA loaded into the membrane. Half of the samples were then exposed to UV light, at which point, the samples were frozen, lyophilized, and reconstituted in deuterated chloroform for NMR analysis. The results of this NMR study are shown in FIG. 6. As the figure shows, only in the case where there is DMPA loaded into the membrane and the polymersomes are exposed to UV light, is there conversion of the acrylate groups (disappearance of peak “a”). The NMR sample for the DMPA-loaded, UV-exposed polymersomes was also significantly more viscous than the other three and had to be diluted in order to obtain a quality spectrum, which is further evidence of polymerization. Additionally, the exposure time was relatively short (5 min) for complete acrylate conversion, limited any photobleaching or degradation of encapsulated compounds. For comparison, 30 min of UV exposure was not sufficient to convert the acrylate groups if no DMPA was present.

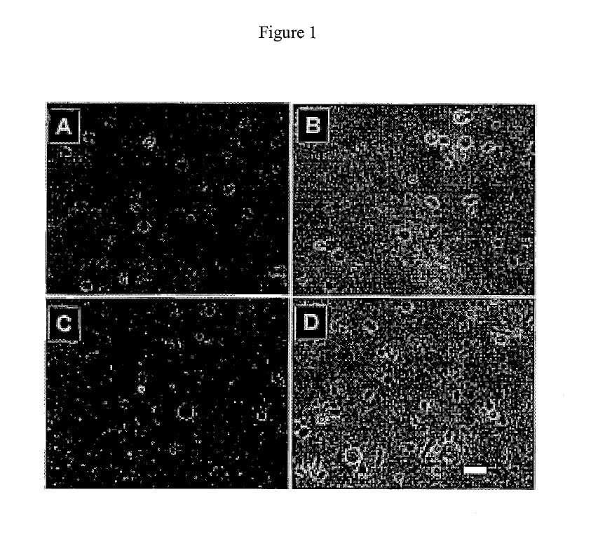

Formation of Giant Polymersomes. Giant polymersomes spontaneously assemble when agitated following an extended incubation at elevated temperatures. To ensure that polymersome structure was not affected by the loading of DMPA into the membrane at high concentrations or by exposure to intense UV light, giant polymersomes were imaged in phase contrast with and without DMPA loaded into the membrane before and after a 30 min exposure to UV light. These images are shown in FIG. 1. As is clear from the images, DMPA loading and UV light had no significant effect on polymersome morphology.

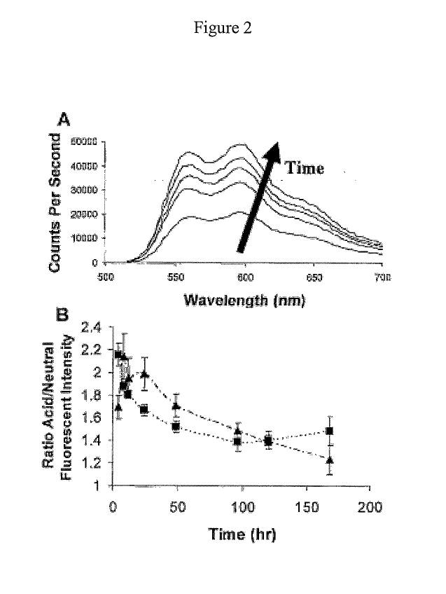

Release of Doxorubicin. Prior studies have shown that DOX initially releases by passive diffusion across the polymersome membrane at neutral pH, while the release is dominated by membrane breakdown at decreased pH. In order to investigate if the membrane of polymersomes with the polymerized acrylates is stabilized into a more inhibitory structure, the release of DOX from polymersomes was monitored over time as a function of pH. These polymersomes break down into units of CL, PEG, and kinetic chains of poly(acrylic acid). When inside the polymersome core, DOX complexes with the ammonium sulfate and forms a gel-like precipitate that self-quenches its fluorescence. Upon release from polymersomes, the fluorescence increases and can be quantified (FIG. 2a). The peak fluorescence of polymersome suspensions over a 7 day period were measured to quantify the release behavior of DOX at neutral and acidic pH.

The relative fluorescence intensity of acidic and neutral samples of acrylated, shell-stabilized polymersomes (DMPA-loaded, UV-exposed) were directly compared to unstabilized polymersomes comprised of non-acrylated PCLPEO (FIG. 2b). While an initial burst is seen in the release at both neutral and acidic pH for both formulations (data not shown), the acid/neutral ratio of the burst intensity was greater for the shell-stabilized polymersomes, and the ratio of release continued to be greater for the shell-stabilized polymersomes for at least the first four days of incubation. These results indicate that the pH differential release from the stabilized polymersomes is much higher than for non-polymerized polymersomes Without being bound to any one theory of operation, this may indicate that early passive diffusion is lower in these newer systems compared to degradation mediated release.

Summary of Results

This work demonstrated that particular modification of end-group chemistry could slow undesirable release of drug from biodegradable polymersomes without significantly altering any other properties of the vesicle.

### Second Example Embodiment

To minimize the release of a drug from polymersomes prior to membrane hydrolysis, it was sought to decrease the release rate of DOX by stabilizing the membrane structure. Specifically, the terminal hydroxyl end-group of the PCL block of the diblock polymer was acrylated prior to polymersome formation. With the addition of an initiator and light source, the acrylates underwent free-radical polymerization and stabilized the polymersome membrane. While not affecting overall polymersome morphology, the polymersomes became more resistant to surfactant disruption and exhibited a decreased release rate of encapsulated DOX at physiological pH.

### Experimental

Materials. Except where noted, all materials were used as received. Acryloyl chloride, triethylamine (TEA, anhydrous grade), poly(ethylene glycol) methyl ether (PEG, Mₙ=2,000 Da), stannous octoate, ε-caprolactone (CL), 2,2-dimethoxy-2-phenylacetophenone (DMPA), TritonX-100, and doxorubicin hydrochloride (DOX) were purchased from Sigma Aldrich. CL was dried for 48 hours over calcium hydride and distilled under reduced pressure immediately prior to use. PEO was dried under vacuum at 90° C. overnight before use. All solvents were reagent grade or better.

Polymer Synthesis. The functionalized polymer was produced in two steps (Scheme 1). In the first, the diblock copolymer was synthesized as previously reported utilizing monomethoxy PEG as a macroinitiator for the ring-opening polymerization of caprolactone with stannous octoate as the catalyst. Briefly, 12g CL (caprolactone) (11.65 mL) was added to a 100 mL round-bottomed flask containing 2.0 g of the previously-dried PEG under an argon atmosphere. 12 drops of stannous octoate were added, and the flask was sealed. The reaction was heated to 90° C. for 30 minutes to fully dissolve the PEG in the CL, and then heated to 130° C. for 1.5 hrs while under vacuum. The crude polymer was dissolved in THF, precipitated into hexanes, and dried overnight. The resulting polymer contained an inert methoxy cap on the PEG and a reactive hydroxyl at the PCL terminus. The hydroxyl terminus of the PCL was acrylated using acryloyl chloride and TEA. 5.2 grams of PCL-PEG were dissolved in 200 mL dichloromethane (DCM) under a stream of argon in a 500 mL 3-neck round-bottomed flask equipped with an addition funnel. 500 μL (˜10×) TEA was injected into the flask and the temperature was reduced to 0° C. The addition funnel was charged with 300 μL (˜10×) acryloyl chloride and 30 mL DCM. The acryloyl chloride solution was added dropwise to the reaction vessel over 1 hour. The reaction was allowed to proceed for 4 hrs at 0° C. followed by overnight at room temperature. Pure polymer was recovered by concentration, dissolution in benzene (to precipitate triethylammonium salts), filtration, reconcentration in DCM, precipitation into hexanes, and drying. Upon purification, the polymer was characterized by NMR and GPC. NMR spectra were recorded on a Bruker Avance 360 MHz spectrometer in deuterated chloroform. GPC spectra were obtained on a Waters 1525 Binary HPLC equipped with an autosampler, refractive index detector and Styragel HR 4E and 5E columns in series utilizing THF as the mobile phase.

Polymersome Formation and Characterization. Polymersomes were fabricated by the self-assembly of thin films of polymer from roughened Teflon into aqueous medium (70-100 mg/ml solution of polymer in dichloromethane, drying, immersion in aqueous solution), followed by sonication at 65° C., freeze-thaw cycling (5 cycles liquid nitrogen to 65° C.), and heated, automated extrusion (400 and 200 nm membranes). The photoinitiator 2,2-dimethoxy-2-phenylacetophenone (DMPA, 18 μg/mg polymer for 1/1 mol/mol) was co-cast for inclusion into the membrane and UV light exposure was completed with an OmniCure Series 1000 spot-curing lamp with a collimating lens (Exfo, Ontario, Canada; 365 nm, 55 mW/cm²). DOX was encapsulated utilizing an ammonium sulfate gradient (280 μL of 10 mg/mL DOX in water was added to 2 mL of a 14 mg/mL polymersome suspension and incubated at 65° C. for seven hours), and free DOX was removed on 2 HiTrap desalting columns in series (GE Healthcare). Release into PBS was monitored by recording the fluorescence of polymersome suspensions over time (SPEX Fluorolog-3 fluorimeter, λₑₓ=480 nm, λₑₘ=590 nm). The amount of DOX encapsulated was determined by polymersome dissociation with addition of 100 μl of 30% TritonX-100 and incubation for 60 min at 37° C. Polymersomes were analyzed for acrylate conversion via NMR and GPC of dehydrated samples reconstituted in organic solvents.

Dynamic light scattering (DLS) measurements were made in PBS or acetate buffer (pH=5, 290 milliosmolal) on a Malvern Zetasizer NanoS (Malvern Instruments, Southboro, Mass.). Samples were prepared by a 20× dilution of the polymersome suspension into the appropriate buffer. TritonX-100 solutions were prepared prior to the addition of the polymersomes, such that the addition of the polymersomes brought the Triton to a final concentration of 0.5 volume percent. These samples were stirred at 37° C. for one hour before transfer to cuvettes. Samples that kept for long-term degradation studies (>1 day) were capped and sealed in their cuvettes to prevent evaporation.

Cryo-TEM images were obtained on a JEOL 1210 TEM. All grids for cryo-TEM were prepared within a controlled environment vitrification system (CEVS) in a saturated water vapor environment at 25° C. A droplet (˜10 μL) of sample was placed on a carbon-coated copper TEM grid (Ted Pella) held by non-magnetic tweezers. Filter paper was used to blot excess sample away, resulting in a thin film of solution spanning the grid. The sample was allowed to relax for approximately 30 s to remove any residual stresses imparted by blotting, then quickly plunged into liquefied ethane (˜90 K) cooled by a reservoir of liquid nitrogen to ensure the vitrification of water. Prepared grids were stored under liquid nitrogen until imaging. The imaging of the grids was executed at −178° C. using a Gatan 626 cryogenic sample holder in a JEOL 1210 TEM operating at 120 kV. A cooled Gatan 724 multiscan CCD camera was used to record the images. Image processing, including background subtraction, was completed with Gatan Digital Micrograph software version 3.9.1.

Cytotoxicity Analysis. NIH 3T3 fibroblasts (ATCC, cultured in DMEM with 10% fetal bovine serum, 1% sodium bicarbonate, 1% pen/strep) were seeded at a density of 10,000 cells/well in a 24-well plate. After 24 hours, the cells were washed and incubated with a suspension of polymersomes in 9:1 media:PBS. The suspensions were sterilized with 20 minutes exposure to a germicidal lamp prior to addition to cells. After 24 or 72 hours of polymersome exposure, the cells were washed and incubated for 4 hours with a 10% solution of Alamar Blue (Biosource, Camarillo, Calif.) in media. The media was removed from the cells and a 100 μL aliquot was read for fluorescence on a plate reader (Bio-Tek Synergy HT). For cytotoxicity studies with DOX-loaded polymersomes, the polymersomes were prepared as described above. To prevent DOX leakage, these polymersomes were kept on ice until immediately before dilution into media and feeding to cells.

Statistics. A Student's t-test was used to determine statistical significance between cell viability groups. A p value of <0.05 was considered significant.

Results and Discussion

Provided were design stabilized polymersomes that were also biodegradable. To that end, a functional group (i.e., acrylate) was incorporated at the PCL terminal end of PCL-PEG diblock polymers. Once assembled into polymersomes and in the presence of a photoinitiator, UV light exposure induces a radical polymerization through the functional groups, as shown in Scheme 2. This approach did not hinder hydrolysis of the PCL chain and yields oligo-caprolactone units, PEG, and kinetic chains of poly(acrylic acid) as the degradation products.

During self-assembly into polymersomes, it is expected that the individual polymer backbones align with each other, forming the membrane. Without being bound to any single theory of operation, it was postulated that despite the semi-crystalline nature of the PCL block within the membrane which could hinder molecular movement, the acrylate groups would be aligned with a close enough proximity to undergo free-radical polymerization upon initiation. To test this hypothesis, a hydrophobic radical photoinitiator (DMPA) was loaded into the bilayer of acrylated PCL-PEG (AcPCL-PEG) polymersomes.

FIG. 6 illustrates NMR spectra of samples made according to the claimed invention; lowercase letters indicate assignment of peaks to the chemical structure shown, FIG. 6 (A) shows NMR spectra of dehydrated polymersomes of AcPCL-PEG with or without DMPA loaded into the membrane before and after UV light exposure as indicated. The −DMPA+UV sample received a 30 minute dose of UV light, while the +DMPA+UV sample received a 5 minute dose. FIG. 6 (B) shows NMR spectra of AcPCL-PEG polymersomes with varying amounts of DMPA loaded into the membrane. All samples received at 10 minute dose of UV light.

Only in the case where DMPA was loaded into the bilayer and the polymersomes were exposed to UV irradiation was polymerization of the acrylate groups observed (i.e., disappearance of acrylate peaks in NMR spectra, FIG. 6A). Additionally, significant peak broadening can be seen in the NMR spectrum of the UV-exposed polymersomes containing DMPA, also indicative of an increase in molecular weight that would be expected to accompany acrylate polymerization. UV light alone or simply the presence of DMPA were both insufficient to induce polymerization.

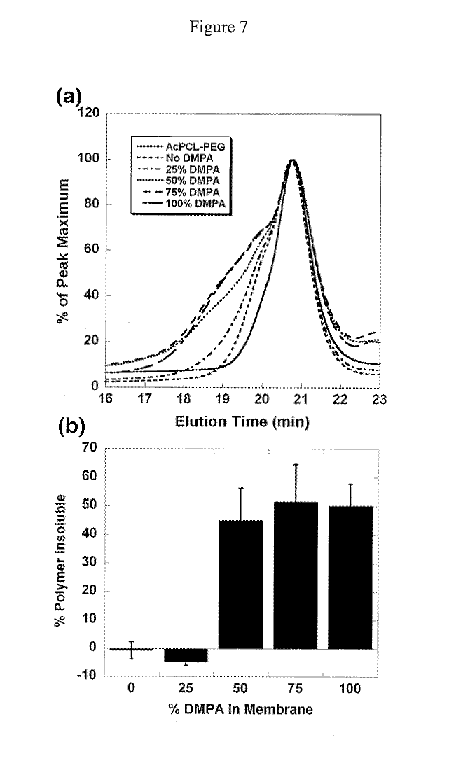

The amount of DMPA necessary for complete conversion of the acrylate groups was also investigated (FIG. 6B and FIG. 7). A 1:1 mol:mol ratio of DMPA to polymer was necessary for complete conversion of acrylates, while partial conversion was observed in the NMR spectra for lower amounts of DMPA. Significant peak broadening was observed in the GPC traces of dehydrated polymersomes reconstituted in THF that had been loaded with between 50 and 100 mol % DMPA. Furthermore, in all of these cases, approximately half of the polymer remained insoluble in THF for GPC analysis, due to a large molecular weight resulting from chain linkage, indicative of polymerization within the membrane. Again without being bound to any single theory of operation, the DMPA was expected to be fairly immobile within the membrane, as the PCL was well below its melting temperature of ˜60° C. Without being bound to any single theory, while though such a high dose of DMPA is required, only those DMPA molecules that have assembled near the acrylates would actually initiate polymerization.

Without being bound to any particular theory of operation, varying the relative amount of DMPA to acrylate (i.e., reactive groups) in the reaction mixture effects some measure of control over the degree of stabilization within the final polymersome product. Thus, by varying the relative amount of DMPA to reactive group so as to achieve the largest average chain length, the user gives rise to polymersomes having a high degree of stabilization, which in turn translates into polymersomes that degrade at a comparatively slow rate.

By altering the amount of DMPA—or other initiator—in the reaction mixture to give rise to comparatively shorter polymer chains, the user can reduce the degree of stabilization and produce polymersomes that degrade relatively more quickly, which may be useful in applications where a more rapid—but controlled—delivery of an agent may be desirable.

FIG. 7 depicts (a) GPC traces of reconstituted polymersomes containing varying amounts of DMPA in the membrane and exposed to UV light. The GPC traces include only the soluble portions of the THF samples. FIG. 7(b) depicts the percentage of polymer that remained insoluble during reconstitution in THF. As shown in that figure, increasing the amount of DMPA, up to a point, increased the relative amount of insoluble polymer.

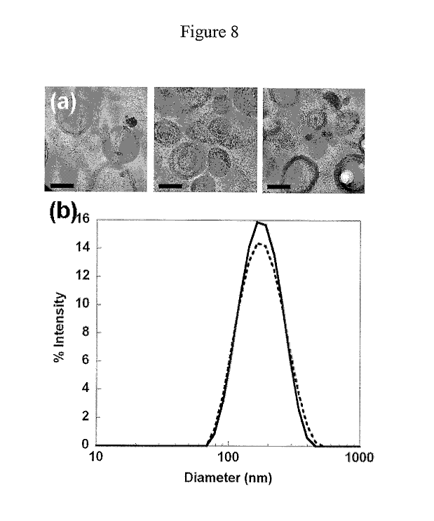

To ensure complete acrylate conversion, a 1:1 ratio of DMPA to polymer was used in subsequent studies. However, it was first confirmed that the high loading of DMPA did not significantly affect the structure of the membrane or resulting macromolecular assembly. Polymersomes of AcPCL-PEG on both the nano-scale and the micro-scale with and without DMPA loaded in the membrane were fabricated (FIG. 1). A robust yield of polymersomes was observed via cryo-TEM (FIG. 8a) with no significant change in size or shape. The polymersomes appeared to be multi-lamellar (FIG. 8). This phenomenon could, without being bound to any particular theory of operation, be allayed by increasing the number of freeze-thaw cycles or passes through the extrusion membrane during processing. Furthermore, to alleviate concerns that a high dose of free radicals in the membrane could lead to polymer cleavage and result in micelle formation, DLS was performed on DMPA-loaded polymersomes before and after UV irradiation (FIG. 8b). The peak for samples before and after light exposure is centered at ˜200 nm and there is no change with light exposure, confirming that micelles do not form. Therefore, while the 1:1 ratio of DMPA to polymer was comparatively high, it did not affect the resulting macromolecular structure.

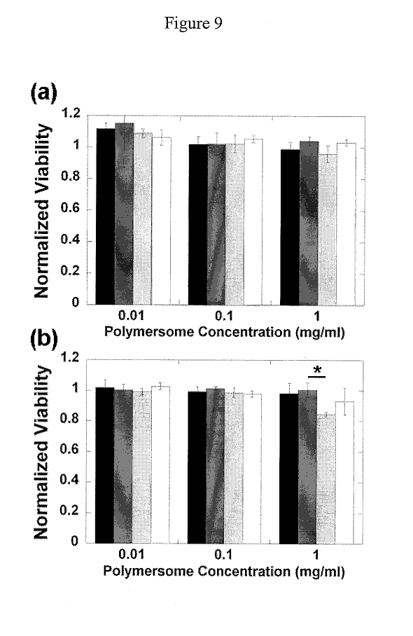

To eliminate any concerns about the biocompatibility of the new polymer chemistry or inclusion of photoinitiator, the potential cytotoxicity was investigated with fibroblasts in standard cultures for up to 72 hours (FIG. 9). A slight drop in viability (˜5-10%) was observed with the AcPCL-PEG+DMPA formulation prior to stabilization, and then only at the highest concentration compared to PBS controls. This decrease in viability was eliminated when the same polymersomes were stabilized by exposure to UV light, suggesting that the toxicity is caused by the high amount of DMPA present. Thus, there were no concerns with the toxicity of UV-stabilized polymersomes, which ultimately degrade into components commonly used in the biomaterials field.

While DMPA was studied in producing the exemplary embodiments described herein, other compositions capable of initiating polymerization are also suitable. For those applications that entail disposition of stabilized polymersomes into a patient or subject, initiators—such as DMPA—that result in biocompatible polymerization products are preferable. A non-exhaustive listing of such initiators includes, for example, DMPA and Irgacure-2959 (Ciba). UV-sensitive initiators, such as DMPA, phenanthrenequinone, and camphorquinone, are considered suitable for the inventive compositions and methods because such initiators are well-characterized. Other initiators, such as those initiators sensitive to heat, i.e. AIBN or ammonium persulfate, or redox, i.e., benzoyl peroxide and N,N-dimethyl-p-toluidine, environments are also suitable.

In some embodiments, the use of an initiator may not be necessary. In these embodiments, the polymer chain materials and any reactive groups thereon are chosen so as to be capable of effecting polymerization without addition of an initiator. In such embodiments, polymerization may be effected by environmental conditions in the vicinity of the polymer chain materials.

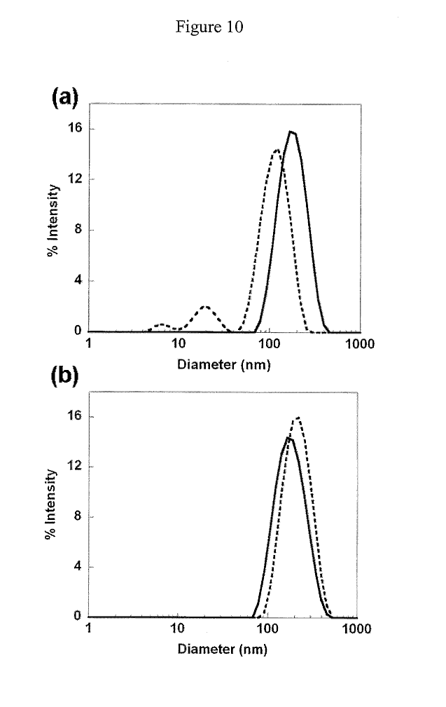

In order to confirm that a physical stabilization had occurred within the polymersome membranes, samples were treated with the detergent Triton X-100 and examined by DLS to check for morphological changes. The changes in size distributions are shown in FIG. 10. For DMPA-loaded samples that were not exposed to UV light (FIG. 10a), a second peak appears around 20 nm and there is a shift of the main peak from ˜200 nm to ˜100 nm after exposure to the detergent (the peak around 8 nm is from free Triton). However, for polymersomes exposed to UV light (FIG. 10b), no micelle peak appears in the DLS and there is a slight increase in the peak diameter of the polymersomes, suggesting that the Triton is partitioning into the membrane, but is unable to cause a complete disruption. Thus, there appears to be enhanced stabilization in polymersomes with polymerization of the acrylates within the membrane.

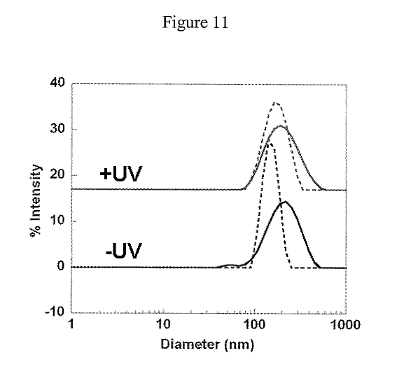

It was also useful to determine whether these polymersomes remained biodegradable. FIG. 11 demonstrates the decrease in polymersome size following incubation for twelve days in acetate buffer at 37° C. During this period of time, the size and dispersity of the polymersomes decreased, indicative of degradation of the hydrophobic (PCL) backbone in the membrane. While a shift is seen for samples both before and after UV exposure, it is clearly more pronounced for the non-exposed sample. As PCL is known to degrade slowly, it is not surprising that over this period of time, only small changes in the polymersome size are observed. However, this data still indicates that as time progresses, the polymersomes degrade.

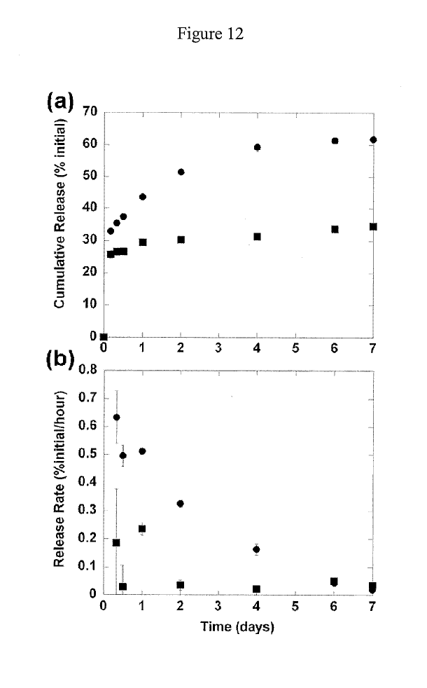

To prove that stabilization of polymersome membranes is useful, a clinically-relevant anti-cancer drug (DOX) was encapsulated in AcPCL-PEG polymersomes loaded with DMPA in the membrane and the release was monitored via fluorescence de-quenching of DOX. Formulations with and without 15 minutes exposure to UV light were compared (FIG. 12a).

As DOX releases from the polymersome and is diluted into the surrounding solution, its fluorescence increases over a baseline level, enabling tracking of the release from the polymersomes. There is existing interest in improved formulations of DOX that slow and target its release, as DOX is known for side effects, as well as exhibiting fast metabolism and degradation at physiological pH. Results are normalized to the initial amount of DOX encapsulated (determined by membrane disruption through Triton exposure to an additional sample for each group) less the baseline fluorescence. Formulations were also highly stable, exhibiting negligible release (<1%) when stored at 4° C. over the same period of time.

For both stabilized and non-stabilized polymersomes, a large initial burst of release was observed; this release was slightly larger for the non-stabilized polymersomes. Without being tied to any particular theory, this burst could be from either the large osmotic gradient or by DOX that partitioned into the membrane prior to stabilization (DOX is amphiphilic). However, following the initial burst, the rate of release was much lower for stabilized polymersomes compared to the non-stabilized (FIG. 12b). By seven days, only ˜5% more of the DOX was observed to be released following the burst, compared to ˜25% more being released for the non-stabilized samples, similar to what was observed previously. It should be noted that because of the degradation of DOX released into solution, exact release profiles cannot be elucidated by this method. However, it is clear from the two observed profiles that release is significantly hindered by stabilization of the membrane.

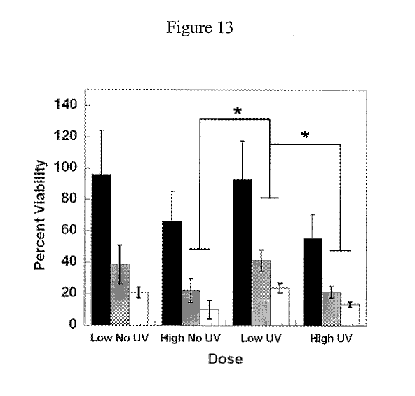

As a final proof of concept, fibroblasts were cultured in the presence of DOX-loaded polymersomes (1.22 or 12.2 mg/ml) and their viability was measured for up to 72 hours (FIG. 13). The concentration of DOX loading in the polymersomes was calculated to be 25% based on the UV absorbance of a diluted sample exposed to Triton after separation on HiTrap columns (ε=23 cm²/mg). As seen in FIG. 13, the viability dropped significantly for cells treated with DOX-loaded polymersomes in both a time- and dose-dependent manner. After 24 hours, viability ranged from ˜55% for the high dose to over 95% for the lower dose. However, by 72 hours, viability dropped to ˜10 to ˜20% for all doses. This in vitro study confirmed that the UV-stabilization did not affect the ability of the DOX to adversely affect the cells once released.

In addition to the agent-delivery applications previously discussed (which applications include, inter alia, delivery of therapeutic agents and imaging agents), the claimed invention may also be suitable for altering the mechanical properties of a polymer membrane, polymersome, and the like. For certain applications, an unstabilized membrane may not be suitable because it lacks the necessary rigidity or toughness, but a membrane that comprises cross-links may be effectively too tough such that it does not degrade in a way that promotes controlled, sustained release of agents disposed within the membrane or within the interior of the polymersome. For these applications, an unstabilized membrane may be stabilized so as to lend some mechanical rigidity to the structure, giving rise to a membrane that degrades over time instead of either quickly degrading (unstabilized membrane) or degrading too slowly or not at all (cross-linked membrane).

Summary of Results

Disclosed are stabilized polymersomes that remain biodegradable following polymerization. Their ease of preparation and low cytotoxicity make this system a candidate for in vitro and in vivo use, as does the polymersomes' payload capacity. The decreased release rate observed for stabilized vesicles is significant for situations in which a high, site-specific dose is required for treatment. Future modifications could involve designing multiple degradable linkages at the hydrophobic terminus of the block copolymer, allowing for enhanced stabilization, or incorporating additional functionality into the acrylated polymer or onto the polymersome, such as PEG-surface modification, to enable active in vivo targeting.

What is claimed:

1. A method of synthesizing a stabilized polymersome, comprising:
forming a polymersome, having a layer structure, from chains of multiblock copolymer comprising hydrophobic and hydrophilic blocks;
at least some of the hydrophobic blocks comprising one or more polymerizable groups; and
reacting two or more of the polymerizable groups to form covalent bonds between chains of the copolymer.

2. The method of claim 1, wherein the multiblock copolymer comprises a diblock copolymer, a triblock copolymer, or any combination thereof.

3. The method of claim 2, wherein the multiblock copolymer comprises poly(caprolactone)-poly(ethylene glycol).

4. The method of claim 1, wherein the reacting comprises reacting two or more of the polymerizable groups in the presence of an initiator.

5. The method of claim 1, wherein the reacting is effected by an environmental condition exterior to the multiblock copolymer.

6. The method of claim 1, wherein the layer structure comprises a bilayer.

7. The method of claim 6, wherein the reacting gives rise to covalent bonds between the individual polymers comprising the bilayer.

8. The method of claim 1, wherein the initiator is 2,2-dimethoxy-2-phenylacetophenone.

9. The method of claim 4, wherein reacting the polymerizable groups comprises exposing the polymerizable groups to ultraviolet light, heat, or both in the presence of the initiator.

10. The method of claim 1, wherein the polymerizable group comprises an acrylate, methacrylate, acrylamide, methacrylamide, vinyl, or vinyl sulfone unit.

11. The method of claim 1, further comprising disposing one or more agents within the stabilized polymersome, within the layer structure, on the surface of the polymersome, or any combination thereof.

12. The method of claim 11, wherein the agent comprises a therapeutic composition, an imaging composition, a binding composition, or any combination thereof.

13. The method of claim 1, wherein the stabilized polymersome is characterized as being biodegradable.

14. The method of claim 1, wherein a majority of the hydrophobic blocks bear a polymerizable group.

15. The method of claim 1, further comprising forming one or more cross-links between multiblock copolymer chains.

16. The method of claim 15, wherein the cross-links are formed by introducing a composition having multiple polymerizable groups to the chains of multiblock copolymer.

17. The method of claim 15, wherein the multiblock copolymer comprises multiple polymerizable groups.

18. The stabilized polymersome made according to claim 1.

19. A stabilized polymersome, comprising:
a polymersome comprising a multiblock copolymer layer structure,
the layer structure comprising multiblock copolymer chains having hydrophilic and hydrophobic blocks, and
two or more chains being covalently bonded to one another.

20. The stabilized polymersome of claim 19, wherein the layer structure is characterized as being a bilayer.

21. The stabilized polymersome of claim 19, comprising two hydrophobic blocks covalently bonded to one another.

22. The stabilized polymersome of claim 19, wherein the stabilized polymersome is characterized as being biodegradable.

23. The stabilized polymersome of claim 19, wherein a hydrophilic block comprises poly(ethylene oxide), poly(acrylic acid), poly(ethylene glycol), or any combination thereof.

24. The stabilized polymersome of claim 19, wherein the hydrophobic block comprises poly(caprolactone), poly(methylcaprolactone), poly(menthide), poly(lactide), poly(glycolide), poly(methylglycolide), poly(dimethylsiloxane), poly(isobutylene), poly(styrene), poly(ethylene), poly(propylene oxide), or any combination thereof.

25. The stabilized polymersome of claim 19, wherein the stabilized polymersome further comprises one or more agents disposed within the layer structure, within the interior of the polymersome, on the surface of the polymersome, or any combination thereof.

26. The stabilized polymersome of claim 25, wherein an agent comprises a drug, a therapeutic compound, a nanoparticle, an imaging agent, a contrast agent, a nutrient, a vitamin, a protein, DNA, RNA, an oligonucleotide, a salt, a gene, a biological material, a magnetic material, a radioactive material, or any combination thereof.

27. The stabilized polymersome of claim 19, wherein the stabilized polymersome is capable of releasing an agent disposed within the polymersome, within the layer structure of the polymersome, or both, at a higher rate when the stabilized polymersome is exposed to a stimulus.

28. The stabilized polymersome of claim 27, wherein a stimulus comprises an acidic environment, an osmotic gradient, oxidative or reductive stress, or heat.

29. The stabilized polymersome of claim 27, further comprising one or more cross-links between one or more multiblock copolymer chains.

30. A method for delivering an agent to a subject, comprising:
introducing one or more stabilized polymersomes into a patient,
the one or more stabilized polymersomes having a layer structure comprising multiblock copolymer, the multiblock copolymer comprising hydrophobic and hydrophilic blocks, the one or more stabilized polymersomes comprising one or more therapeutic agents; and
exposing the one or more stabilized copolymers to a stimulus to effect release of the one or more therapeutic agents.

31. The method of claim 30, wherein the one or more stabilized polymersomes are taken up by one or more cells characterized by a diseased state.

32. The method of claim 30, wherein one or more cells in a diseased state expose the one or more stabilized polymersomes to the stimulus.

33. The method of claim 30, wherein the stabilized polymersome is used to treat a disease, to assist in imaging, or any combination thereof.

34. The method of claim 30, wherein exposing the stabilized polymersome to the stimulus effects an enhanced release of the one or more agents from the stabilized polymersome.

35. A method for altering the properties of a polymersome, comprising:
providing a polymersome,
the polymersome comprising a multiblock copolymer layer structure,
the structure comprising a plurality of multiblock copolymer chains,
the multiblock copolymer comprising hydrophilic and hydrophobic blocks; and
forming covalent bonds between two or more hydrophobic blocks of multiblock copolymer chains.

36. The method of claim 35, wherein the forming of covalent bonds is accomplished by polymerizing reactive groups present on the hydrophobic blocks.

37. The method of claim 35, further comprising forming one or more cross-links between copolymer chains.

38. The method of claim 35, wherein the layer structure comprises a bilayer.
